(19) United States
(12) Patent Application Publication
(10) Pub. No.: US 2026/0194389 A1
(43) Pub. Date: Jul. 9, 2026

(54) OPTICAL MEASUREMENT SYSTEMS AND TECHNIQUES FOR WATER USED IN FOOD AND BEVERAGE PROCESSES

(71) Applicant: Ecolab USA Inc., Saint Paul, MN (US)

(72) Inventors: Richard Joesph Walsh, Woodbury, MN (US); Daniel Arthur Stroberger, Lakeville, MN (US); Rachel McGinness, Rosemount, MN (US); Robert Ryther, Saint Paul, MN (US); Seth Michael Detjens, St. Paul, MN (US)

(21) Appl. No.: 19/440,597

(22) Filed: Jan. 5, 2026

Related U.S. Application Data
(60) Provisional application No. 63/742,256, filed on Jan. 6, 2025.

(51) Int. Cl. G01J 1/02; G01N 1/14; G01N 21/31; G01N 33/18

(52) U.S. Cl. CPC G01J 1/0252; G01J 1/0238; G01N 1/14; G01N 21/314; G01N 33/18; G01N 2201/023

(57) ABSTRACT

A method of controlling an optical sensor exposed to a high-fouling water source from a food or beverage production operation can include introducing clean water into a flow chamber of the optical sensor. The optical sensor can measure an absorbance of the clean water and a cleanliness value can be determined based on the measured absorbance. If the cleanliness value is beyond a cleanliness threshold, a deep cleaning is performed on the optical sensor. Otherwise, if the cleanliness value is within the cleanliness threshold, a measurement sample from the food or beverage production operation can be introduced into the optical sensor and a concentration of an antimicrobial agent in the measurement sample determined.

### RELATED APPLICATION

This application claims the benefit of and priority to U.S. Provisional Application No. 63/742,256, filed on Jan. 6, 2025, the entire contents of which are incorporated herein by reference.

### TECHNICAL FIELD

This disclosure relates to optical measurement systems and techniques and, more particularly, to optical measurement systems and techniques for monitoring and/or controlling water used in food and beverage production processes.

### BACKGROUND

Water is used throughout commercial food and beverage production operations, e.g., to manufacture food or beverage products, to convey food products or ingredients along a production line, and/or to clean equipment used in the production process. Cleaning and/or antimicrobial agents may be added to various water streams used in food and beverage production operations. Commercial users rely upon the concentration of a cleaning or antimicrobial product to make the product work effectively. Failure of a cleaning or antimicrobial product to work effectively (for example due to concentration issues) can cause production problems and lead a commercial user to perceive the product as lower quality. In addition, commercial users may be investigated and/or sanctioned by government regulatory and health agencies if operating without the required level of cleaning and/or antimicrobial chemistry. For these and other reasons, a user may monitor the characteristics of a fluid solution, e.g., to determine if the concentration of a product is within a specified concentration range.

### SUMMARY

In general, this disclosure is directed to optical sensor systems and optical-based techniques for measuring a concentration of one or more chemical species in water used in a production process, such as the concentration of a cleaning and/or antimicrobial agent. The example systems and techniques can be implemented in applications where the water subject to analysis contains solids and/or contaminants that have a tendency to foul the optical sensor, presenting challenges with accurately and reproducibly monitoring the comparatively high-fouling water due to sensor fouling. For instance, in one application, the described optical sensor systems and optical-based techniques can be used to analyze water in a food or beverage production operation. The water subject to analysis may be water used to convey solid food articles along a production line, water used to clean production line equipment, or other water used in the food or beverage production facility. In any case, the water may contain one or more contaminants that can form a fouling layer on and/or over optical windows of the optical sensor analyzing the water. The foulant can build up on the optical windows through repeated exposure to the water being analyzed, interfering with the optical measurements made using the optical sensor.

In accordance with some examples of the present disclosure, systems and techniques are described for optically analyzing a comparatively high-fouling water source, such as that from a food or beverage production operation, while maintaining the accuracy and reproducibility of measurements made by the optical sensor. In some application, the described systems and techniques are implemented to determine a cleanliness associated with the optical sensor and to determine of the optical sensor is becoming fouled. If it is determined that the optical sensor is not sufficiently clean, a deep cleaning can be executed on the optical sensor before subsequently using the optical sensor to analyze a water sample to determine a concentration of one or more chemical species of interest in the water.

In some examples, an optical sensor system is implemented to measure a concentration of a chemical agent in a water source used in a food and/or beverage production operation. For example, the chemical agent of interest may be an antimicrobial agent introduced into a production water used to convey food items along a production line in which the food items are cleaned and/or processed. The antimicrobial agent may prevent and/or inhibit the formation of microbial contaminants in the water, helping to keep the water sanitary and the food items being processed unaffected by microbial growth and contamination.

In operation, the optical sensor can receive a sample of water from the water source for analysis. One or more reagents may be added to the water sample, such as a colorimetric reagent and/or one or more pH adjusting agents. The resulting treated water sample can then be optically analyzed by the optical sensor to generate an optical response (e.g. absorbance) corresponding to the amount of the chemical species of interest in the water sample. Accordingly, by comparing the magnitude of the optical response generated from the water sample under analysis to calibration information relating known optical responses to known concentrations of the chemical species of interest, the concentration of the chemical species of interest can be determined.

To determine if the cleanliness of the optical sensor is deteriorating to a point where deep cleaning of the optical sensor is desired, systems and techniques of the disclosure may be implemented to measure a cleanliness value associated with the optical sensor. To measure the cleanliness value, clean water different than the process water being analyzed can be introduced into the flow chamber of the optical sensor. The optical sensor can then make an absorbance measurement with the clean water in the flow cell. The absorbance measurement can be used to determine a cleanliness value for the optical sensor. For example, the measured absorbance with clean water in the flow cell can be compared to a reference absorbance measured by a reference detector, where light emitted by the optical sensor and detected by the reference detector does not pass through the clean water. The cleanliness value can be determined based on comparison of the absorbance determined by the reference detector to the measured absorbance through the clean water sample. If the cleanliness value is beyond a cleanliness threshold, a deep cleaning can be performed on the optical sensor. By contrast, if the cleanliness value is within the cleanliness threshold, the optical sensor may proceed to analyze the process water to determine the concentration of one or more chemical species of interest.

In some examples, systems and techniques according to the disclosure operate with a regular cleaning of the optical sensor after analysis of each process water system to help prevent foulant buildup and prolong the period of time before a deep cleaning is needed on the optical sensor. For example, after each optical analysis of each process water sample using the optical sensor, the flow cell of the optical sensor may be flushed of the sample and a cleaning agent introduced into the flow cell. The cleaning agent may be held in the flow cell until analysis of the next process water sample. In either case, contamination may still build up in the flow cell overtime notwithstanding the regular cleaning performed on the flow cell. Accordingly, a cleanliness value for the optical sensor may be analyzed and used to control a more extensive deep cleaning on the optical sensor as described herein.

In one example, a method of controlling an optical sensor exposed to a water source from a food or beverage production operation is described. The method includes introducing clean water into a flow chamber of an optical sensor, measuring an absorbance of the clean water using the optical sensor to provide a measured absorbance, and determining based on the measured absorbance a cleanliness value for the optical sensor. The example method also includes comparing, by one or more processors, the cleanliness value to a cleanliness threshold and, if the cleanliness value is beyond of the cleanliness threshold, performing a deep cleaning on the optical sensor. By contrast, the method specifies that if the cleanliness value is within the cleanliness threshold the method may proceed to introduce a measurement sample into the flow chamber of the optical sensor that includes a process water from a food or beverage production operation have an antimicrobial agent. The method may also include measuring an absorbance of the measurement sample to provide a measurement sample absorbance and determining a concentration of the antimicrobial agent based on the measurement sample absorbance.

In another example, an optical sensor system is described that includes at least one water pump, at least one reagent pump, an optical sensor, and a controller. The at least one water pump is configured to be fluidly connected to a source of a clean water and a source of process water from a food or beverage production operation that includes an antimicrobial agent. The at least one reagent pump is configured to be fluidly connected to a source of a reagent and to introduce the reagent into a sample of the process water supplied by the at least one water pump. The optical sensor includes a flow chamber, a light emitter, a reference detector configured to detect light emitted from the light emitter without passing through the flow chamber, and a measurement detector configured to detect light emitted from the light emitter passing through the flow chamber. The controller is communicatively coupled to the water pump, the reagent pump, and the optical sensor. The example specifies that the controller is configured to control the water pump to introduce the clean water into the flow chamber of the optical sensor, control the optical sensor to emit light via the light emitter that is detected via the reference detector to provide a reference absorbance and detected via the measurement detector to provide a measurement absorbance, and determine, based on comparison of the reference absorbance to the measurement absorbance, a cleanliness value for the optical sensor.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram of an example food and beverage production operation that can utilize an optical sensor arrangement according to the disclosure.

FIG. 2 is a block diagram of an example configuration of an optical sensor system that can be used in the system of FIG. 1.

FIG. 3 is a flow diagram illustrating an example technique that can be executed by the optical sensor system of FIG. 2 to determine a cleanliness value for the optical sensor and when to perform a deep clean on the optical sensor.

FIG. 4 is a diagram of an example system implementation of the optical sensor system of FIG. 2

### DETAILED DESCRIPTION

Fluids with chemical agents are used in a variety of different industries for a variety of different applications. For example, in the food and beverage production industry, water that includes an antimicrobial agent can be used to convey food items being processed, such as a fruit, a vegetable, and/or a meat. For instance, a poultry production facility may use a conveyance line having a flowing stream of water to convey cut meat to be washed and/or for downstream processing and packaging. An antimicrobial agent may be introduced into the water used on the conveyance line to inhibit microbial growth in the water due to contamination from the food items being processed. Ensuring that the water has a proper concentration of antimicrobial agent can help ensure that the water is safe and effective for the production process. For example, the concentration of antimicrobial agent may be set and maintained between certain high and low concentration threshold values to help ensure the quality and safety of the water used in the production process.

This disclosure describes optical sensor systems and techniques utilizing an optical sensor to monitor the concentration of a chemical agent (e.g., antimicrobial agent) in a water, such as a food and/or beverage process water. Depending on the application, the optical sensor may be implemented as an online sensor that receives a flow of water from a source of the process water on a continuous or periodic basis and analyzes the water to determine the concentration of the chemical agent in substantially real-time. For example, the optical sensor may be connected to a flow of process water from the food and beverage production facility via a pipe, tube, or other conduit. The optical sensor may then receive a sample of the process water from the source via the conduit and analyze the water to determine the concentration of the antimicrobial agent in the water. The concentration of the antimicrobial agent can be adjusted in the process water based on the analysis.

In practice, the process water analyzed by the optical sensor may contain a number of contaminants that have a tendency to foul the optical sensor. If undetected and untreated, the fouling can result in inaccurate measurements regarding the amount of antimicrobial agent in the water being analyzed. This can lead to underdosing or overdosing of the antimicrobial agent in process water, diminishing the effectiveness of the chemical treatment program.

In accordance with aspects of the present disclosure, the optical sensor system can be implemented to determine a cleanliness value for the optical sensor. The cleanliness value can be indicative of fouling build up in the optical sensor. If the cleanliness value for the optical sensor is at an acceptable level, the optical sensor can be used to determine the concentration of the antimicrobial agent in the process water being analyzed. However, if the cleanliness value for the optical sensor outside of an acceptable level, a deep cleaning may instead be performed on the optical sensor. In this way, the optical sensor can be reliably implemented in a high fouling environment while ensuring that measurements made by the optical sensor and corresponding control actions are accurate and reliable.

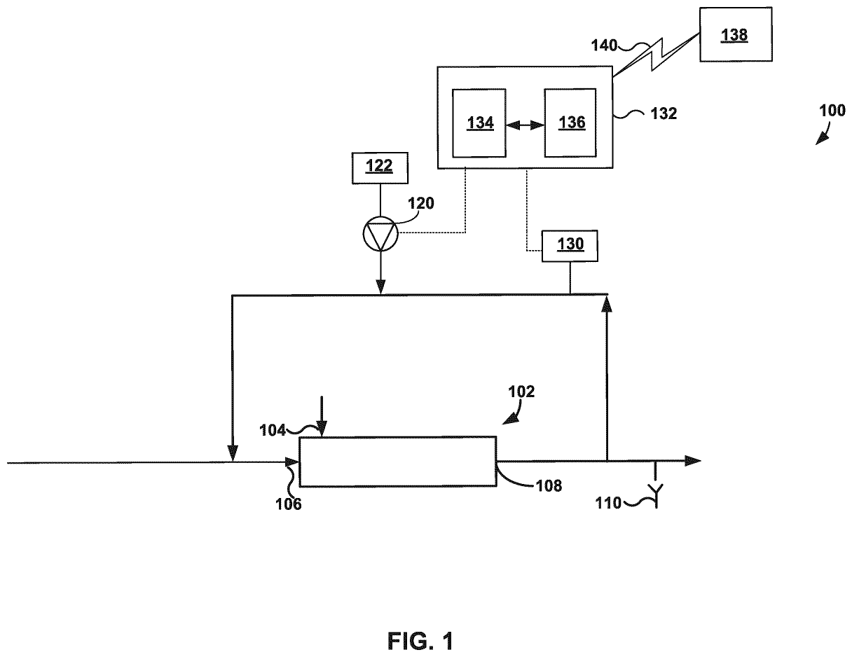

FIG. 1 is a conceptual diagram of an example food and beverage production operation 100 that can utilize an optical sensor arrangement according to the disclosure. In the illustrated example, production operation 100 includes one or more processing regions 102, which is conceptually illustrated as a single processing region, that receives a food and/or beverage component and contacts the food and/or beverage component with water. Processing region 102 may be an area of production operation 100 configured to wash a food item being processed, e.g., by spraying, immersing, and/or otherwise contacting the food item with water. Additionally or alternatively, processing region 102 may be a conveyance line configured to convey a food item being processed from one area of the operation to another area of the operation via a flowing stream of water.

Independent of the specific configuration of production operation 100, the production operation may include a process water that includes water having contacted a food item being processed. For example, in the illustrated arrangement, one or more food items being processed can enter processing region 102 at a food inlet 104 and be contacted with water entering via a water inlet 106. The one or more food items being processed may remain substantially stationary while being contacted with the water or may move in a conveyance direction with a flowing stream of water. The food items can discharge processing region 102 at a food outlet 108. Depending on the arrangement, water contacting the food items in processing region 102 can discharge the processing region separately from the food items (e.g., by draining through the food items and exiting through a separate water outlet) or can exit processing region 102 with the food items being processed and be subsequently separated.

In production operation 100, a wide variety of fruits, vegetables, and/or meats may be washed and/or conveyed, e.g., to ensure cleanliness and safety before packaging or further processing. Common fruits that undergo washing include apples, oranges, grapes, berries (such as strawberries, blueberries, and raspberries), bananas, pears, peaches, pineapples, mangos, plums, avocados, lemons, limes, kiwis, pomegranates, tomatoes, cherries, and melons like cantaloupe, watermelon, and honeydew. Vegetables such as lettuce, spinach, kale, cabbage, kale, collard greens, carrots, potatoes, cucumbers, peppers, broccoli, cauliflower, zucchini, eggplant, asparagus, green beans, celery, mushrooms, beets, and onions are also routinely washed to remove dirt, pesticides, and contaminants. While tomatoes are technically a fruit, they are often treated as a vegetable and washed alongside other vegetables.

In addition to produce, meats are similarly washed to maintain hygiene. Common meats that undergo washing in a commercial plant include chicken (whole or in parts like breasts, thighs, and wings), beef (such as steaks, ground beef, and roasts), pork (chops, ground pork, and ribs), lamb, turkey (whole or in parts), fish (like salmon, tuna, and cod), shellfish (such as shrimp, lobster, and crab), sausages, bacon, and duck. These meats are often rinsed or soaked in water, with an antimicrobial agent, to remove contaminants like blood, bone fragments, or other impurities. The washing processes for food items (e.g., fruits, vegetables, meats) in a food production plant can help ensure that the products are safe for consumption and meet food safety regulations.

While FIG. 1 illustrates one example configuration of production operation 100 that can generate a process water optically analyzed according to systems and techniques of the disclosure, the described systems and techniques can be used in a variety of different environments for a variety of different fluids. Example process waters may be those used in any food processing application, including those used in or for washing, rinsing, preserving, sanitizing, disinfecting, sterilizing, harvesting, peeling, cutting, blanching, scalding, cooking, chilling, irrigating, flavoring, pickling, soaking, marinating, curing, coating, transporting, floating, separating, sorting and/or disposing food processing application.

In some implementations of production operation 100, the residual process water after use is discarded to a drain 110. In these applications, the process water is used as a single contact or on a once-through basis. In other applications, some or all of the residual process water after use is recycled for reuse on another batch of one or more food items. For example, the used process water may be recovered after it has been used in a food processing application. The used process water can be recovered from any of a number of desired sources including, but not limited to, food transport water, food wash water, food sanitizing/disinfecting/sterilizing water, conveyor belt wash water, conveyor sanitizing/disinfecting/sterilizing water, equipment wash water, equipment sanitizing/disinfecting/sterilizing water, package wash water, food packaging rinse water, package sanitizing/disinfecting/sterilizing water, and combinations thereof. In some examples, the process water is recovered from one or more chiller tanks. The process water may be water that that contacts a food item directly or water that contacts equipment used to process food. For example, the process water may be water used to clean the process equipment (without food items being present) and may include one or more cleaning and/or sanitizing agents.

The process water recovered for reuse may include dissolved or undissolved components from the food items contacting the water. Residual food components present in the process water may include, but are not limited to, blood, non-food debris, feathers, hairs, twigs, pebbles, organic chemical compounds, inorganic chemical compounds, fats, oils, and greases. The process water may be processed in a variety of ways to help remove residual food components before reuse, including using a separation method such as filtration, centrifugation, flotation, flocculation, coagulation, purging, and combinations thereof.

In either case, one or more chemical agents may be added to the process water used in production operation 100. In the configuration of FIG. 1, production operation 100 includes one or more pump 120 fluidly connected to one or more respective chemical reservoirs 122. Pump 120 can operate to add one or more chemicals to the process water used in production operation 100 that contacts one or more food items. In some examples, an antimicrobial agent may be added to the water, in which case pump 120 may be referred to as an antimicrobial agent pump. Any antimicrobial agent suitable for use in the desired end use application can be used. In some examples, one antimicrobial agent is used. In other embodiments, two, three or more antimicrobial agents are used. Example antimicrobial agent or agents can include, but are not limited to: peroxygen compounds, ozone, chlorine dioxide, acidified sodium chlorite, chlorine, chlorine releasing agents, bromine releasing agents, quaternary ammonium compounds, cetylpyridinium chloride, organic acids, and mixtures thereof.

In certain implementations, the antimicrobial agents may be selected from the chemical class of oxidizing chemistries. Oxidizing antimicrobial agents can include, without limitation, chlorine-containing species and there in situ or equilibrated forms, such as hypochlorous acid, hypochlorite, chlorous acid, chlorite, chlorine dioxide, dichloroisocyanurates, and related chlorine-releasing systems; hydrogen peroxide; and peroxyacids that may be used alone or in combination with hydrogen peroxide, such as peroxyacetic acid, peroxyoctanoic acid, peroxyformic acid, peroxypropionic acid, peroxyheptanoic acid, and mixtures thereof. These oxidizers may be provided as single-component actives, multi-component blends, or precursor systems that generate the active species in situ, and may be formulated with pH adjusters, buffers, catalysts, stabilizers, surfactants, sequestrants, and other excipients to tailor antimicrobial efficacy and compatibility with the optical measurement protocol described herein.

In some examples, a peroxygen compound is used as the antimicrobial agent. Peroxygen compounds that may be used can include, but are not limited to, peroxyacetic acid, peroxyoctanoic acid, peroxyformic acid, peroxypropionic acid, peroxyheptanoic acid, peroxybenzoic acid, peroxynonanoic acid, monoperglutaric acid, diperglutaric acid, succinylperoxide, hydrogen peroxide, and mixtures thereof. In some examples antimicrobial solution used is a peroxyacid mixture including acetic acid, octanoic acid, hydrogen peroxide, peroxyacetic acid, and/or peroxyoctanoic acid.

In some applications, an antimicrobial agent is selected from a group of chlorine or non-chlorine halogen compounds including, but not limited to: iodines, iodophors, bromines, brominated compounds, and mixtures thereof. Additionally or alternatively, an antimicrobial agent may be selected from a group of quaternary ammonium compounds including, but not limited to: quaternary ammonium chlorides, cetylpyridinium chloride, and mixtures thereof. Organic acids (e.g., lactic acid, citric acid, propionic acid), mineral acids (e.g., phosphoric acid, hydrochloric acid, sulfuric acid), and mixtures thereof may also be used. In still yet other examples, an antimicrobial agent may include sodium metasilicate, potassium metasilicate, and mixtures thereof.

The antimicrobial agent can be selected to kill or inhibit the growth of a variety of microorganisms, including bacteria, fungi, parasites, protozoa and combinations thereof. Microorganisms that may be present in the process water and treated with the antimicrobial agent can include, but are not limited to: Acinetobacter, Aeromonas, Alcaligenes, Bacillus, Campylobacter, Clostridium, Enterococcus, Flavobacterium, Lactococcus, Lactobacillus, Leuconostoc, Listeria, Micrococcus, Moraxella, Pediococcus, Pseudomonas, Shewanella, Staphylococcus, Vibrio, Streptococcus, Salmonella, Escherichia, Citrobacter, Enterobacter, Erwinia, Klebsiella, Proteus, Serratia, Shigella, Yersinia, Alternaria Aspergillus, Aureobasidium, Botrytis, Byssochlamys, Cladosporium, Fusarium, Geotrichum, Mucor, Penicillium, Rhizopus, Candida, Cryptococcus, Rhodotorula, Saccharomyces, Trichosporon, Zygosaccharomyces, Picorvaviruses, Reoviruses, Parvoviruses, Papovaviruses, Adenoviruses, Rotaviruses, Hepatitis A virus, Norwalk virus, Giardia, Entamoeba, Cryptosporidium, Toxoplasma, flatworms, roundworms, and combinations thereof.

In the illustrated configuration of FIG. 1, pump 120 is illustrated as adding chemical additive to process water being recycled for reuse. In practice, the chemical additive may be introduced at any suitable location. Moreover, while production operation 100 in FIG. 1 illustrates a single pump fluidly coupled to a single chemical additive reservoir, the pump may be in selective fluid communication with multiple reservoirs containing different chemicals and/or the system may include multiple pumps each configured to introduce a different chemical into the process water.

To ensure that an appropriate amount of one or more chemical agents, such as an antimicrobial agent, are present in the process water in production operation 100, the system may include at least one optical sensor system 130 as described in greater detail below. Optical sensor system 130 can be implemented to optically analyze a sample of the process water and determine therefrom a concentration of one or more chemical agents of interest in the water. This information can then be used in a feedback to control addition of the one or more chemical agents of interest in the water, e.g., ensuring that the concentration of the one or more chemical agents of interest are controlled within a desired concentration range/ threshold in the process water.

Optical sensor system 130 can be implemented in number of different ways in production operation 100. For example, one or more of the sensors can be positioned in line with process water flowing through processing region 102 (e.g., upstream or downstream of the processing region) either directly or via a slipstream pulled from the main flowing water stream. Alternatively, one or more of the sensors may be implemented as an off-line monitoring tool that is not in direct fluid communication with water flowing through systems. In in these applications, process water may be extracted from production operation 100 and transported to an off-line analysis system. Such off-line analysis may involve evaluation of the sample using an offline implementation of optical sensor system 130. In either case, data generated by optical sensor system 130 can be received by a controller 132, e.g., for storage in memory and/or further processing.

Production operation 100 in the example of FIG. 1 includes controller 132. Controller 132 can be communicatively connected to optical sensor system 130 and controllable components of production operation 100 to manage the overall operation of the system. For example, controller 132 can be communicatively connected to pump 120, optical sensor system 130, and/or any other controllable components in the system (e.g., one or more valves to control addition of fresh makeup water, discharge of process water to drain 110).

Controller 132 includes a processor 134 and memory 136. Controller 132 communicates with communicatively connected components via a wired and/or wireless connection, which in the example of FIG. 1 is illustrated as a wired connection. Control signals sent from controller 132 and received by the controller can travel over the connection. Memory 136 stores software for running controller 132 and may also store data generated or received by processor 134, e.g., from optical sensor system 130. Processor 134 runs software stored in memory 136 to manage the operation of production operation 100.

Controller 132 may be implemented using one or more controllers, which may be located at the facility site of production operation 100. Controller 132 may communicate with one or more remote computing devices 138 via a network 140. For example, controller 132 may communicate with a geographically distributed cloud computing network, which may perform any or all of the functions attributed to controller 132 in this disclosure.

Network 140 can be configured to couple one computing device to another computing device to enable the devices to communicate together. Network 140 may be enabled to employ any form of computer readable media for communicating information from one electronic device to another. Also, network 140 may include a wireless interface, and/or a wired interface, such as the Internet, in addition to local area networks (LANs), wide area networks (WANs), direct connections, such as through a universal serial bus (USB) port, other forms of computer-readable media, or any combination thereof. On an interconnected set of LANs, including those based on differing architectures and protocols, a router may act as a link between LANs, enabling messages to be sent from one to another. Communication links within LANs may include twisted wire pair or coaxial cable, while communication links between networks may utilize analog telephone lines, full or fractional dedicated digital lines, Integrated Services Digital Networks (ISDNs), Digital Subscriber Lines (DSLs), wireless links including cellular and satellite links, or other communications links. Furthermore, remote computers and other related electronic devices may be remotely connected to either LANs or WANs via a modem and temporary telephone link.

In operation, optical sensor system 130 can generate data indicative of a concentration of one or more chemical compounds present in the process water being analyzed. Controller 132 can receive data from the optical sensor system and use a determined concentration measurement for one or more chemical agents of interest to control addition the one or more chemical agents from reservoir 122 to the process water via pump 120.

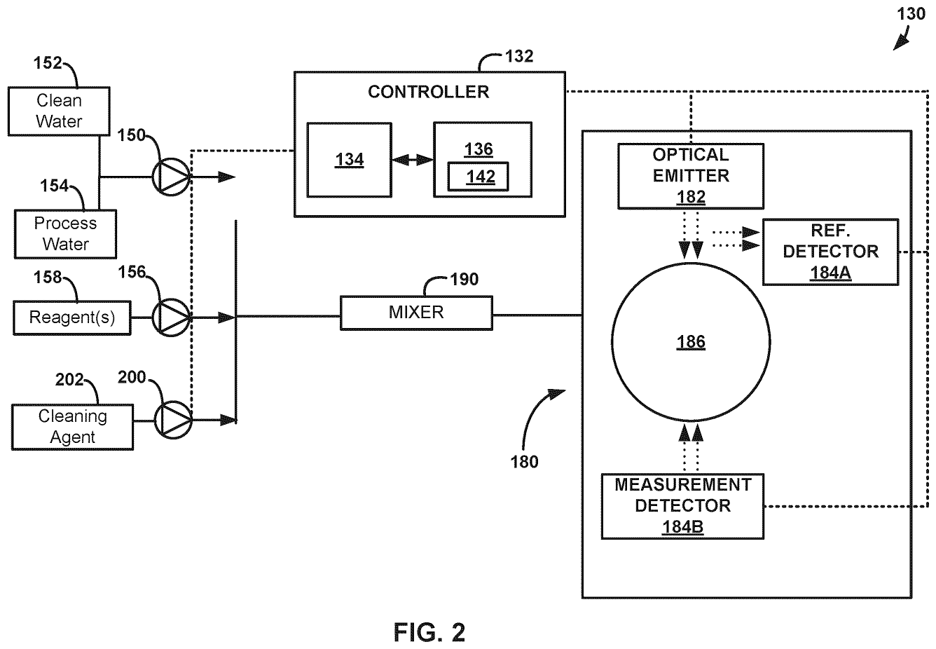

FIG. 2 is a block diagram of an example configuration of optical sensor system 130 that includes an optical sensor that may be exposed to a comparatively high-fouling water source, such as from a food or beverage production operation. Optical sensor system 130 is illustrated in the form of a system that includes an optical sensor including an emitter and an optical detector as well as hardware for extracting a process water sample and mixing one or more reagents with the sample prior to optical analysis. In other configurations, optical sensor system 130 may be implemented without the reagent and mixing features of FIG. 2 or may have other configurations than the specific example illustrated. Moreover, optical sensor system 130 in FIG. 2 is illustrated as being controlled by controller 132 discussed above with respect to FIG. 1. Controller 132 can be communicatively connected to the controllable components within optical sensor system 130 to send and/or receive data and control signals to/from the components. However, the functions described as being performed by controller 132 in FIG. 2 (e.g., with respect to determining a cleanliness value) may be performed by a different system controller, one or more remote computing devices 138, and/or other controller hardware and/or software.

In the example of FIG. 2, optical sensor system 130 is illustrated as including at least one water pump 150 configured to be fluidly connected to a clean water source 152 and a process water source 154 (e.g., from food and beverage production operation 100), which can contain one or more chemical species desirably measured, such as an antimicrobial agent. Optical sensor system 130 may include multiple water pumps, including one or more fluidly connected to each of clean water source 152 and process water source 154 or may include a single water pump fluidly connected to both clean water source 152 and process water source 154 and that is in selective communication with each source by controlling one or more valves. In either case, water pump 150 can operate to provide a sample of process water from process water source 154 containing an unknown concentration of a chemical species of interest, such as an unknown concentration of an antimicrobial agent. Water pump 150 can also operate to provide clean fluid for flushing through the fluid conduit(s) of optical sensor system 130 and/or filling a flow chamber of the optical sensor with clean water.

Optical sensor system 130 can also include a reagent pump 156 fluidly connected to a reagent source 158. Operating under the control of controller 132, reagent pump 156 can pump one or more reagents from reagent source 158 to be introduced into and combined with process water from process water source 154 supplied by water pump 150. A variety of different reagents can be supplied to combine with process water supplied by water pump 150. In some examples, the reagent is a colorimetric reagent (e.g., an indicator) that can complex or otherwise react with a chemical species of interest (e.g., an antimicrobial agent) to produce a measurable optical response, the extent of which varies in response to the amount of the chemical species of interest present in the sample under analysis. One example colorimetric reagent that can be used is potassium iodide (KI), which can react with an oxidizing antimicrobial agent to produce a colorimetric response.

In some examples, optical sensor system 130 is configured to introduce one or more additional or different reagents to a process water sample undergoing optical analysis. The one or more additional reagents may be present with colorimetric reagent and introduced simultaneously with the colorimetric reagent or may be introduced separately from the colorimetric reagent (using reagent pump 156 or a different reagent pump). Other example chemical reagents that may be added to the process water sample in addition to or in lieu of a colorimetric reagent include, but are not limited to, a pH adjuster and/or buffer, a reaction catalyst, a sequestrant, a surfactant, and/a combination thereof. For example, controller 132 may control the addition of a pH adjustor to the process water sample undergoing analysis so the process water sample is within a pH range where the colorimetric reagent forms a colorimetric response with the chemical species being measured. In some examples, controller 132 controls addition of an amount of acid to the process water sample effective to ensure that substantially all (e.g., all) of an oxidizing antimicrobial agent present in the process water sample is in acid form and can react with the colorimetric reagent (e.g., potassium iodide) to produce a colorimetric response.

In some implementations, the colorimetric determination of oxidizing antimicrobial chemistry is carried out by introducing iodide (e.g., potassium iodide) into the sample stream, whereby the iodide is oxidized by the oxidizing antimicrobial(s) to iodine and rapidly establishes the I−/I2/I3− equilibria, yielding triiodide. In process waters sourced from foods that do not contain starch, such as poultry or other non-starchy meats, the measurement can be performed without adding starch and in accordance with the methods described herein, because the triiodide itself provides a sufficient optical signal at the selected wavelength(s). However, in process waters associated with starchy foods, such as corn or potatoes, some fraction of starch may become solubilized into the process water.

In such cases, an initial background measurement may be performed by dosing iodide alone to characterize the baseline absorbance attributable to triiodide-starch complex arising from oxidizer present in the absence of additional starch. A subsequent analytical measurement can then be conducted with iodide and an added amount of solubilized starch (e.g., a defined aliquot of starch solution or a controlled release from a reservoir) to ensure the triiodide-starch complex reaches a reproducible color response that scales with oxidizer concentration. The controller can correct the analytical measurement using the background to account for native starch contributions in the process water, thereby providing an accurate determination of the oxidizer level across both starch-free and starch-containing food processing applications.

To measure the optical response of the process water sample (e.g., when combined with one or more reagents such as a colorimetric reagent), optical sensor system 130 includes an optical sensor 180. Optical sensor 180 includes one or more optical emitters 182 and one or more optical detectors 184 optically connected to a flow chamber 186 the provides a space for receiving a sample for optical analysis. Flow chamber 186 may be an optical cell that receives and holds a static portion of fluid that undergoes optical analysis, for example, in a stop flow configuration with the sample subsequently being discharged. As another example, flow chamber 186 may be or include a fluid conduit through which a flowing stream of fluid passes with optical analysis being performed on the flowing stream of fluid.

Optical sensor 180 may include an optical window between the sample receiving space defined by flow chamber 186 and optical emitter 182 and an optical window between the sample receiving space and optical detector 184. Each optical window may be a clear or transparent section fluid conduit defining the flow chamber and/or may be or include a lens (e.g., ball lens) providing a barrier between the fluid being analyzed and the optical emitter and detector. Each optical window may be formed of any suitable material (e.g., plastic, glass, sapphire), and may define a surface in contact with the liquid in flow chamber 186. As discussed above, over time, contaminants in the process water from process water source 154 may have a tendency to build up on the optical windows, impacting the optical response of the process water sample under analysis being measured by optical sensor 180.

During operation, controller 132 can control optical sensor system 130 to provide a sample of process water from process water source 154 (e.g., by controlling water pump 150) and combine the process water sample with one or more reagents from reagent source 158 (e.g., by controller reagent pump 156). One reagent combined with the process water sample may be a colorimetric reagent that reacts with a chemical species of interest (e.g., an antimicrobial agent) to provide a colorimetric response, the intensity of which varies in proportion to the concentration of the chemical species of interest in the process water sample. The process water sample and one or more reagents can be combined upstream of optical sensor 180 (e.g., in a manifold) and may pass through a mixer 190 (e.g., static mixer) as the combined liquids flow to optical sensor 180, helping to ensure uniform mixing of the different liquids. In other configurations, the process water sample and one or more reagents can be combined at optical sensor 180, e.g., by being introduced into different ports fluidly connected to flow chamber 186. The reaction between a colorimetric reagent and an antimicrobial agent may therefore occur in or upstream of flow chamber 186 or in the flow chamber and be detected by measuring an optical response (e.g., absorbance) of the combined sample.

In one example, operating under the control of a controller (e.g., controller 132), an automated concentration measurement process may be executed in which a predetermined amount of process water from process water source 154 is combined with a predetermined amount of a colorimetric reagent and/or other reagent(s), thereby generating a measurement sample supplied to flow chamber 186. The controller can control optical sensor 180 to measure an optical response of the measurement sample in flow chamber 186, thereby providing a measurement sample optical response (e.g., a measurement sample absorbance). The controller can determine a concentration of a chemical species of interest in the process water sample (e.g., the antimicrobial agent) based on the measurement sample optical response (e.g., with reference to calibration information stored in memory 136 relating different optical responses to different concentrations of the chemical species of interest).

As mentioned above, optical sensor 180 can include one or more optical emitters that emit light into each sample at one or more wavelengths and one or more detectors that detect light from the fluid sample at one or more wavelengths, which may be the same as or different than the wavelengths emitted into the fluid sample. In different examples, the one or more optical detectors may detect light passing through the fluid sample under analysis (e.g., transmittance, absorbance), light scattered by the fluid sample, a fluorescent response of the fluid sample, or yet other optical response of the fluid sample. In one specific example, optical sensor is configured to detect the absorbance of the fluid sample under analysis. As is known by those of ordinary skill in the art, absorbance is a measure of how much incident light is absorbed when it travels in a sample while transmittance measures how much of the light is transmitted through the sample. According, an optical sensor according to the disclosure may measure absorbance and/or transmittance to provide the same function.

Operating under the controller of controller 132, optical emitter 182 can direct light into a fluid sample in flow chamber 186. Optical emitter 182 may include at least one optical emitter that emits radiation over one or more wavelengths, such as a single wavelength or across a specified wavelength range. In some examples, optical emitter 182 emits radiation over continuous range of wavelengths. In other examples, optical emitter 182 emits radiation at one or more discrete wavelengths. For example, optical emitter 182 may emit at two, three, or more discrete wavelengths. Example light sources that can be used as optical emitter 182 include one or more light emitting diodes (LEDS), lasers, and/or lamps.

The specific wavelength(s) over which optical emitter 182 emits light into the fluid sample under analysis may vary depending on the specific colorimetric reagent and/or other reagents combined with the process water sample. In some examples, optical emitter 182 is configured to emit light over a plurality of wavelengths at or near different absorbance maxima for a complex formed between a colorimetric reagent and an antimicrobial species in the process water under analysis. In some examples, optical emitter 182 includes an LED that emits light at a wavelength or within wavelength range from 400 to 500 nm (e.g., to measure absorbance when the antimicrobial species is within a concentration from 0 to 100 ppm), an LED that emits light at a wavelength or within wavelength range from 500 to 600 nm (e.g., to measure absorbance when the antimicrobial species is within a concentration from 100 to 1000 ppm), and/or an LED that emits light at a wavelength or within wavelength range from 600 to 700 nm (e.g., to measure absorbance when the antimicrobial species has a concentration greater than 1000 ppm).

Optical detector 184 can receive light emitted by optical emitter 182. In some implementations, optical sensor 180 includes multiple optical detectors, including a reference detector 184A configured to detect light emitted from optical emitter 182 without passing through flow chamber 186 and a measurement detector 184B configured to detect light emitted from optical emitter 182 and passing through the flow chamber. Reference detector 184A can function to measure the amount of light emitted by optical emitter 182 without signal attenuation due to the contents of the fluid sample in flow chamber 186, fouling on optical windows, the material forming the flow chamber and/or optical windows, and the like.

Light emitted by optical emitter 182 and propagating through the fluid sample in flow chamber 186, fouling on optical windows, the material forming the flow chamber and/or optical windows, and the like can be detected by measurement detector 184B. The amount of radiation detected by optical detector 184B depends on the contents of the fluid sample under analysis. If the sample has a certain concentration of a chemical species of interest (e.g., antimicrobial agent), optical detector 184B can detect a certain level of radiation emitted from optical emitter 182. However, if the sample has a different concentration, optical detector 184B may detect a different level of radiation emitted from optical emitter 160.

Each optical detector 184 can detects radiation over one or more wavelengths emitted by optical emitter 182. The wavelengths detected by optical detector may encompasses the same wavelength or wavelength ranges emitted by optical emitter 182 or may be a broader or narrower range than the wavelength(s) emitted by the optical emitter. When performing absorbance and/or transmittance measurements, each optical detector 184 may detect the same wavelength(s) of light emitted into the sample by optical emitter 182. Each optical detector 184 may include one or more photodetectors such as, e.g., photodiodes or photomultipliers, for converting optical signals into electrical signals.

Controller 132 controls the operation of optical emitter 182 and receives signals concerning the amount of light and/or frequency or wavelength(s) of light detected by optical detector 184. In some examples, controller 132 processes signals received from optical detector 184 during analysis of a process water sample containing an unknown concentration of a chemical species of interest (e.g., an antimicrobial agent) and determines a concentration of chemical species in the process sample based on calibration data 142 stored in memory.

A memory 136 associated with controller 132 (e.g., contained within or physically remote from and providing data accessible to a processor) may store data 142 representative of calibration information (e.g., in the form of a calibration curve, equation, look-up table) used by controller 132 to determine a concentration of a chemical species of interest in the process water sample under analysis. For example, calibration data 142 may relate light detected by optical detector 184B (e.g., a measurement sample absorbance) to a concentration of an antimicrobial agent in the process water sample under analysis. This can provide a determined concentration of the chemical species of interest, such as a determined concentration of the antimicrobial agent.

Controller 132 may compare the determined concentration of one or more chemical species of interest to one or more concentration thresholds and control the introduction of the chemical species of interest into the process water from which the process water sample was extracted. For example, controller 132 may compare a concentration of an antimicrobial agent in the process water sample determined based on a measurement sample absorbance to a concentration threshold stored in memory 136 (e.g., a low concentration threshold and/or a high concentration threshold). The one or more concentration thresholds can be received from a user via a user interface associated with controller 132. Controller 132 can control the amount of antimicrobial agent added to the process water based on the measured concentration and comparison.

For example, controller 132 can control pump 120 (FIG. 1) to increase the amount of the antimicrobial agent added to the process water if the measured concentration is below low concentration threshold. Controller 132 can control pump 120 to start the pump or to increase a dispensing rate of the pump. Additionally or alternatively, controller 132 can control pump 120 to decrease the amount of the antimicrobial agent added to the process water if the measured concentration is above a high concentration threshold. Controller 132 can control pump 120 to stop the pump or to decrease a dispensing rate of the pump. Controller 132 may additionally or alternatively control the process by directing at least some process water to drain 110 and/or introducing fresh make up water to the process water to reduce the overall concentration of antimicrobial agent present in the process water.

As initially discussed above, optical sensor system 130 may be used in a comparatively high fouling environment where the water subject to analysis contains solids and/or contaminants that have a tendency to foul the optical sensor, presenting challenges with accurately and reproducibly monitoring the comparatively high-fouling water due to sensor fouling. For example, process water sampled for analysis in optical sensor 180 from production operation 100 may include foulants such as fats and proteins (raw or uncooked) when processing raw meats or foulants such as starch and/or fibrous material (carbohydrate and/or proteinaceous in composition) when processing produce. These and/or other foulants may build up on and/in optical sensor 180 (e.g., flow chamber 186), interfering with optical measurements made on a process water sample using the optical sensor.

In accordance with some examples of the present disclosure, systems and techniques may be implemented to reduce or eliminate optical sensor performance deterioration associated with monitoring and optically analyzing a comparatively high fouling process water source. For example, after optically analyzing a process water sample using optical sensor system 130, controller 132 may control the optical sensor system to flush the measurement sample from flow chamber 186 (e.g., and associated fluid lines of optical sensor 180 and/or optical sensor system 130). Controller 132 may control water pump 150 to deliver clean water from clean water source 152 to optical sensor 180. Water pump 150 may deliver clean water under pressure, causing the clean water flow through the various fluid conduits and features of optical sensor system 130, such as a mixing manifold, mixer 190, an optical sensor 180, including flow chamber 186.

The clean water delivered by water pump 150 can purge residual process water and any reagents mixed therewith from the components of the system being flush with the water. This residual waste liquid can be directed to a waste line or other suitable discharge location. Controller 132 may control water pump 150 to deliver an amount of clean water to optical sensor 180 to entirely flush the optical sensor (or at least flow chamber 186 of the optical sensor) of residual process water in any reagents mixed there with or reaction products thereof. While controller 132 has been described in the foregoing description as controlling water pump 150 to deliver clean water under pressure to optical sensor 180, in other applications, controller 132 may control delivery of pressurized clean water to optical sensor system 130 and/or optical sensor 180 without controlling a water pump (e.g., by opening a valve to place a pressurized water source in fluid communication with the components being flushed).

In general, clean water source 152 may be any source of water that does not contain (or contains a reduced amount of) contaminants present in the process water source 154 that can cause fouling in optical sensor 180. Clean water source 152 may be a well or mains water from a private or municipal water system that provides potable water, e.g., suitable for human consumption. Clean water may or may not be chlorinated and/or fluorinated. Clean water may be water received directly from a source (e.g., external source) that has not contacted food or beverage items being processed and that has not otherwise been contaminated prior to being introduced into optical sensor system 130. Clean water may or may not undergo further purification after being received from the source and prior to being introduced into optical sensor system 130, such as filtration, ion exchange, or other purification processes.

After being flushed with clean water, the clean water may be held in optical sensor system 130 and/or optical sensor 180. Alternatively, a cleaning agent may be introduced into optical sensor system 130 and/or optical sensor 180. The cleaning agent may be a clean liquid introduced partially or fully along the fluid flow path process water travels through optical sensor system 130, displacing flushing water along the fluid flow path with the clean liquid.

For example, in the example configuration of FIG. 2, optical sensor system 130 is illustrated as including a cleaning agent pump 200 fluidly connected to a source of a cleaning agent 202. Operating under the control of controller 132, cleaning agent pump 200 can operate to introduce a pressurized cleaning agent from source 202 into the fluid pathways fluidly coupled to optical sensor 180 and partially or fully through the optical sensor (e.g., at least flow chamber 186). The cleaning agent can displace clean flushing water present in the fluid pathways, with the residual flushing water being directed to a discharge location such as a waste line. In some examples, controller 132 controls optical sensor system 130 to introduce a liquid cleaning agent into and/or through fluid conduits of the system and/or optical sensor 180 without first flushing the system with clean water.

A variety of different cleaning agents can be used in the systems and techniques of the present disclosure, and the specific composition of cleaning agent or agents selected may vary based on the types of soil and foulants encountered. In some examples, the cleaning agent includes a compound capable of liberating an active halogen species, such as Cl₂, Br₂, I₂, ClO₂, BrO₂, IO₂, OCl⁻, —OBr⁻ and/or, −OI⁻. Example agents can include, for example, chlorine-containing compounds such as a chlorite, a hypochlorite, chloramine. Halogen-releasing compounds can include, for example, the alkali metal dichloroisocyanurates, chlorinated trisodium phosphate, alkali metal hypochlorites, alkali metal chlorites, monochloramine and dichloramine, and the like, and mixtures thereof can be included. The cleaning agent may also be or include an oxidizing agent such as hydrogen peroxide, peracids, perborates, for example sodium perborate mono and tetrahydrate, sodium carbonate peroxyhydrate, phosphate peroxyhydrates, and potassium permonosulfate, with and without activators such as tetraacetylethylene diamine, and the like. In some applications, the cleaning agent includes a surfactant.

After delivering the cleaning agent to the fluid conduits of optical sensor system 130 and/or optical sensor 180, controller 132 can control optical sensor system 130 to cause the cleaning agent to be held in the components in a substantially non-flowing state for a period of time. This can provide a period of time in which the surfaces contacted by the cleaning agent are allowed to soak, helping to dissolve and remove any foulant accumulated thereon. The period of time may be at least one second, such as at least five seconds, at least 10 seconds, at least 20 seconds, at least 30 seconds, at least one minute, at least two minutes, or at least five minutes. For example, the period of time may range from 30 seconds to 10 minutes, such as from 30 seconds to five minutes. If production operation 100 is off-line, the cleaning agent may remain present in optical sensor system 130 for a longer period of time while the production operation is off-line, such as four hours or more, eight hours or more 12 hours or more, or 24 hours or more.

The cleaning agent can be purged from optical sensor system 130 and/or optical sensor 180 with a clean water flush, an air flush, and/or with a new process water sample being delivered for analysis. In some examples, controller 132 controls the system to flush clean water from clean water source 152 through the fluid conduits containing the cleaning agent before initiating a subsequent measurement on a process water sample.

In some examples, controller 132 controls optical sensor system 130 to introduce cleaning agent from cleaning agent source 202 into the fluid conduits and/or optical sensor 180 after each measurement cycle performed on a sample of process water from process water source 154. For example, each time after optically analyzing a measurement sample formed of process water from process water source 154 and one or more reagents from reagent source 158, controller 132 can control the system to introduce cleaning agent into the system (e.g., after flushing with clean water from clean water source 152). In other examples, controller 132 may control optical sensor system 130 to introduce cleaning agent into the fluid conduits and/or optical sensor 180 on a reduced frequency less than after optically analyzing each measurement sample that includes process water.

Independent if or how controller 132 controls optical sensor system 130 to introduce a cleaning agent after optically analyzing a process water sample to determine a concentration of antimicrobial agent in the sample, the controller may control the system to measure a cleanliness value associated with optical sensor 180. The cleanliness value may provide a measure indicative of accumulating fouling in optical sensor 180, particularly flow chamber 186, notwithstanding any flushing and/or cleaning steps performed on the optical sensor after analyzing a measurement sample. The cleanliness value can provide an indication to controller 132 and/or an operator of optical sensor system 130 when a deep cleaning should be performed on optical sensor 180.

The deep cleaning may be a more rigorous cleaning of surfaces of at least optical sensor 180 (e.g., at least flow chamber 186) that help remove accumulated fouling from surfaces of the sensor that otherwise attenuate light emitted into or received from a sample in flow chamber 186. Accordingly, the amount of light received by a sample in flow chamber 186 from optical emitter 182 after performing a deep cleaning and/or detected by measurement detector 184B from the flow chamber after deep cleaning may be greater than prior to the deep cleaning due to the removal of light attenuating foulant on surfaces through which light is emitted and/or received.

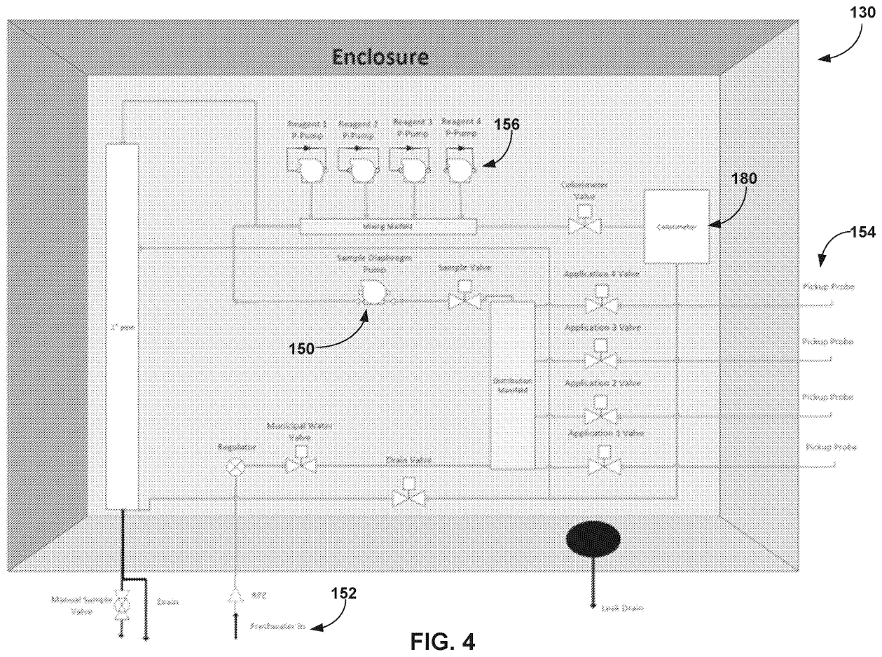

FIG. 4 is a diagram illustrating an example implementation of optical sensor system of FIG. 2 where like reference numerals refer to like features discussed above.

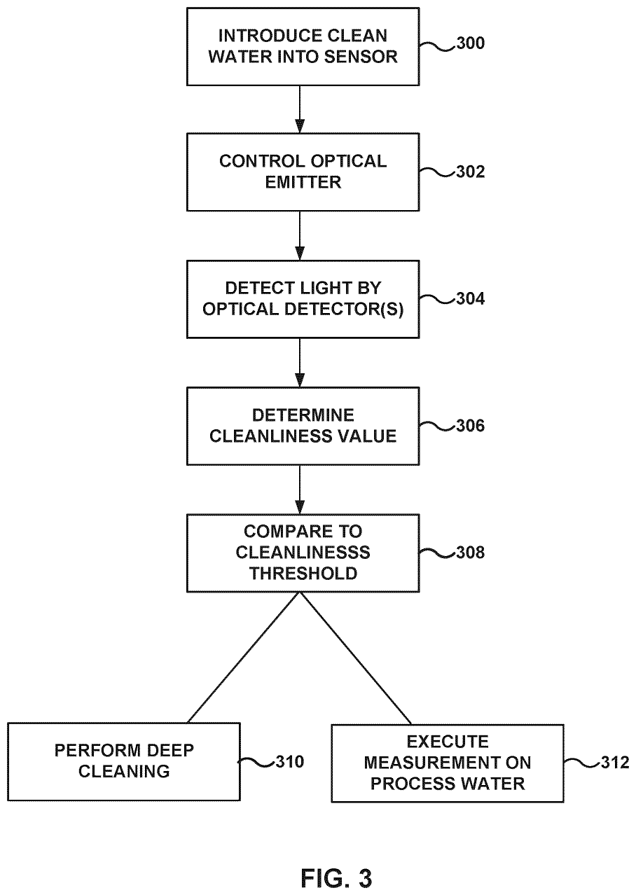

FIG. 3 is a flow diagram illustrating an example technique that can be executed by optical sensor system 130 operating under the control of controller 132 to determine a cleanliness value for optical sensor 180 and to determine when to perform a deep clean on the optical sensor. In the example of FIG. 3, controller 132 controls water pump 150 to introduce clean water from clean water source 152 into flow chamber 186 of optical sensor 180 (step 300). With clean water flowing through or held statically within flow chamber 186, controller 132 can control optical emitter 182 to emit light into and through flow chamber 186 (step 302). Light emitted by optical emitter 182 can be detected by a reference detector 184A without passing through flow chamber 186 or being attenuated by fouling on the surfaces of the flow chamber and can also be detected by a measurement detector 184B after passing through the flow chamber and being attenuated by fouling on the surfaces of the flow chamber (step 304). The amount of light detected by reference detector 184A can provide a reference optical response value (e.g., reference absorbance). The amount of light detected by measurement detector 184B can provide a measurement optical response value (e.g., measurement absorbance).

An example of FIG. 3, controller 132 can determine a cleanliness value for optical sensor 180 based on the measurement optical response determined by measurement detector 184B (step 306). For example, controller 132 can determine the cleanliness value by comparing the reference optical response value (e.g., reference absorbance) to the measurement optical response value (e.g., measurement absorbance). This comparison can provide an indication of how clean optical sensor is and, accordingly, can be deemed the cleanliness value.

Controller 132 can control the reference optical response value to the measurement optical response value in a variety of different ways to determine the cleanliness value. In some examples, controller 132 determines a difference between the reference optical response value in the measurement optical response value, with the difference being the cleanliness value. Additionally or alternatively, controller 132 may determine a ratio of the reference optical response value to the measurement optical response value, with the ratio being the cleanliness value. In general, controller 132 may process the reference optical response value in the measurement optical response value to ascertain the relative difference between the two values, which can indicate the presence and/or relative amount of fouling in optical sensor 180 (e.g., on optical windows of flow chamber 186 of the optical sensor).

In the example of FIG. 3, the example technique involves controller 132 comparing the cleanliness value measured for optical sensor 180 to one or more cleanliness thresholds (step 308). The one or more cleanliness thresholds may be received from a user via user interface and stored in memory 136 associated with controller 132. A cleanliness threshold may be a value that the measured cleanliness value can be compared against to determine when fouling has accumulated to a point that performing a deep cleaning on optical sensor 180 would be appropriate. Controller 132 can compare the cleanliness value measured for optical sensor to a cleanliness threshold and determine if the measured cleanliness value is beyond the cleanliness threshold (e.g., is below the cleanliness threshold or exceeds the cleanliness threshold).

If controller 132 determines that the measured cleanliness value for optical sensor 180 is beyond the cleanliness threshold, a deep cleaning may be performed on optical sensor 180 (step 310). Controller 132 may control optical sensor system 130 to execute the deep cleaning. In some examples, controller 132 executes a deep clean by introducing a cleaning agent into fluid conduits of optical sensor system 130 and/or optical sensor 180, such as at least flow chamber 186 of the optical sensor. The cleaning agent may be the same or may be different than a cleaning agent introduced into the system during regular operation, such as between measurement samples. For example, controller 132 may control cleaning agent pump 200 to introduce cleaning agent from cleaning agent source 202 into the fluid conduits and optical sensor 180. Controller 132 may control the system to hold the cleaning agent in a substantially non-flowing state (e.g., statically) in the system for a longer period of time than the cleaning agent may be held in the system during cleanings during regular operation. For example, controller 132 may hold the cleaning agent in the system, including flow chamber 186, for a period of at least 30 minutes, such as at least one hour, at least two hours, at least four hours, at least six hours, or at least eight hours.

Additionally or alternatively, controller 132 may control optical sensor system 130 to introduce a cleaning agent into the fluid conduits and optical sensor 180, including flow chamber 186 having a different characteristic than the cleaning agent that may be introduced into the system during regular operation. For example, optical sensor system 130 may include a deep cleaning agent separate from cleaning agent 202 that can be delivered by cleaning agent pump 200 or a different pump. The deep cleaning agent may be more aggressive then cleaning agent 202. Additionally or alternatively, the deep cleaning agent may have the same chemical composition as the cleaning agent from cleaning agent source 202 but may have or be used in a higher concentration than the cleaning agent used during regular operation. In this regard, optical sensor system 130 may have a separate source of more concentrated cleaning agent or the concentrate cleaning agent may be diluted or diluted more heavily during regular clean operation.

As still another example, a manual cleaning may be performed on optical sensor 180 (e.g., flow chamber 186) in response to determining that the cleanliness value for the optical sensor is beyond a cleanliness value threshold. In such applications, controller 132 may communicate, directly or indirectly, with a user interface to provide an output to an operator of optical sensor system 130 indicating that a deep clean needs to be performed on the system. In response to receiving an indication via the user interface, an operator may manually access optical sensor 180 and manually clean the optical sensor. For example, an operator may introduce a scouring instrument (e.g., brush, rag) into a lumen of optical sensor 180 and/or flow chamber 186 to remove fouling accumulated on surfaces thereof through mechanical action. In more significant cases, the operator may replace some or all of optical sensor 180 (e.g., flow chamber 186 and/or optical windows thereof) with new components devoid of accumulated fouling.

With further reference to FIG. 3, in instances where controller 132 determines that the measured cleanliness value is within the cleanliness value threshold (e.g., below or above the threshold), the controller may proceed to control optical sensor system 130 to optically analyze a process water sample (step 312). For example, as discussed above, controller 132 may control optical sensor system 130 to deliver an amount of process water from process water source 154 and an amount of one or more reagents from reagent source 158 to be mixed together and delivered to optical sensor 180 for analysis therein.

The techniques described in this disclosure may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. For example, features described as controllers herein such as controller 132 using computing hardware physically co-located with production operation 100 and/or optical sensor system 130 or may be partially or fully physically remote from such features, such as implemented through a remote server, cloud-computing environment, or other physically remote computing device.

The techniques described in this disclosure may also be embodied or encoded in a computer-readable medium, such as a non-transitory computer-readable storage medium, containing instructions. Instructions embedded or encoded in a computer-readable storage medium may cause a programmable processor, or other processor, to perform the method, e.g., when the instructions are executed. Non-transitory computer readable storage media may include volatile and/or non-volatile memory forms including, e.g., random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

1. A method of controlling an optical sensor exposed to a high-fouling water source from a food or beverage production operation, the method comprising:
introducing a clean water into a flow chamber of an optical sensor, measuring an absorbance of the clean water using the optical sensor to provide a measured absorbance, and determining based on the measured absorbance a cleanliness value for the optical sensor;
comparing, by one or more processors, the cleanliness value to a cleanliness threshold;
if the cleanliness value is beyond the cleanliness threshold, performing a deep cleaning on the optical sensor;
if the cleanliness value is within the cleanliness threshold:
introducing a measurement sample into the flow chamber of the optical sensor, the measurement sample comprising a process water from a food or beverage production operation that includes an antimicrobial agent;
measuring an absorbance of the measurement sample to provide a measurement sample absorbance; and
determining a concentration of the antimicrobial agent based on the measurement sample absorbance.

2. The method of claim 1, further comprising, after measuring the absorbance of the measurement sample, flushing the measurement sample from the flow chamber and introducing a cleaning agent into the flow chamber.

3. The method of claim 2, wherein:
introducing the measurement sample, measuring the absorbance, and determining the concentration is performed on multiple different measurement samples to determine the concentration of the antimicrobial agent in each of the multiple different measurement samples; and
flushing the measurement sample from the flow chamber and introducing the cleaning agent into the flow chamber comprises flushing the flow chamber and introducing the cleaning agent into the flow chamber after measuring the absorbance of each of the plurality of different samples.

4. The method of claim 1, wherein:
the optical sensor comprises a light emitter, a reference detector configured to detect light emitted from the light emitter without passing through the flow chamber, and a measurement detector configured to detect light emitted from the light emitter passing through the flow chamber;
measuring the absorbance of the clean water using the optical sensor to provide the measured absorbance comprises:
emitting light by the light emitter;
measuring a reference absorbance by the reference detector; and
measuring the measurement absorbance by the measurement detector; and
determining based on the measured absorbance the cleanliness value for the optical sensor comprises comparing the reference absorbance to the measurement absorbance.

5. The method of claim 4, wherein comparing the reference absorbance to the measurement absorbance comprises determining one or both of a ratio and a difference between the reference absorbance and the measurement absorbance.

6. The method of claim 1, wherein performing the deep cleaning on the optical sensor comprises one or more of:
introducing a concentrated cleaning agent into the flow chamber;
holding a cleaning agent in the flow chamber for a period of at least 30 minutes; and
performing a manual cleaning on the flow chamber of the optical sensor.

7. The method of claim 1, wherein the antimicrobial agent comprises an oxidizing antimicrobial agent.

8. The method of claim 1, further comprising controlling, by one or more processors, an amount of the antimicrobial agent added to the process water based on the concentration of the antimicrobial agent determined based on the measurement sample absorbance.

9. The method of claim 8, further comprising comparing the concentration of the antimicrobial agent determined based on the measurement sample absorbance to a low concentration threshold and a high concentration threshold;
wherein controlling the amount of the antimicrobial agent added to the process water based on the concentration comprises increasing the amount of the antimicrobial agent added to the process water if the concentration is below the low concentration threshold and decreasing the amount of the antimicrobial agent added to the process water if the concentration is below the high concentration threshold.

10. The method of claim 1, wherein the process water comprises water used to clean the food and/or convey the food along a production line.

11. The method of claim 1, wherein the process water is recirculated for multiple cycles along the production line.

12. The method of claim 1, wherein the process water comprises water used in a clean-in-place cleaning process of the food or beverage production operation.

13. The method of claim 1, wherein the measurement sample further comprises a colorimetric reagent, and further comprising mixing the colorimetric reagent with the process water prior to introducing the measurement sample into the flow chamber of the optical sensor.

14. An optical sensor system comprising:
at least one water pump configured to be fluidly connected to a source of a clean water and a source of process water from a food or beverage production operation that includes an antimicrobial agent;
at least one reagent pump configured to be fluidly connected to a source of a reagent and to introduce the reagent into a sample of the process water supplied by the at least one water pump;
an optical sensor that comprises a flow chamber, a light emitter, a reference detector configured to detect light emitted from the light emitter without passing through the flow chamber, and a measurement detector configured to detect light emitted from the light emitter passing through the flow chamber; and
a controller communicatively coupled to the at least one water pump, the at least one reagent pump, and the optical sensor, wherein the controller is configured to:
control the at least one water pump to introduce the clean water into the flow chamber of the optical sensor;
control the optical sensor to emit light via the light emitter that is detected via the reference detector to provide a reference absorbance and via the measurement detector to provide a measurement absorbance; and
determine, based on comparison of the reference absorbance to the measurement absorbance, a cleanliness value for the optical sensor.

15. The system of claim 14, wherein the controller is further configured to:
compare the cleanliness value to a cleanliness threshold; and
if the cleanliness value is beyond the cleanliness threshold, control execution of a deep cleaning on the optical sensor.

16. The system of claim 15, further comprising at least one cleaning agent pump configured to be fluidly connected to a source of a deep cleaning agent, wherein the controller is communicatively coupled to the at least one cleaning agent pump and configured to control execution of the deep cleaning on the optical sensor at least by controlling the at least one cleaning agent pump to supply the deep cleaning agent to the optical sensor.

17. The system of claim 14, wherein the controller is configured, if the cleanliness value is within the cleanliness threshold, to:
control the at least one water pump to supply the sample of the process water;
control the at least one reagent pump to introduce the reagent into the sample of the process water supplied by the at least one water pump to generate a measurement sample;
control the optical sensor to measure the absorbance of the measurement sample to provide the measurement sample absorbance; and
determine a concentration of the antimicrobial agent based on the measurement sample absorbance.

18. The system of claim 17, wherein the controller is configured to:
control the at least one water pump to supply multiple samples of the process water;
control the at least one reagent pump to introduce the reagent into the multiple samples of the process water supplied by the at least one water pump to generate multiple measurement samples;
control the optical sensor to measure the absorbance of each of the multiple measurement samples to provide the measurement sample absorbance for each of the multiple measurement samples; and
determine the concentration of the antimicrobial agent based on the measurement sample absorbance for each of the multiple measurement samples.

19. The system of claim 17, further comprising at least one cleaning agent pump configured to be fluidly connected to a source of a cleaning agent, wherein the controller is communicatively coupled to the at least one cleaning agent pump and configured, after controlling the optical sensor to measure the absorbance of the measurement sample to provide the measurement sample absorbance, to control the at least one cleaning agent pump to supply the cleaning agent to the optical sensor.

20. The system of claim 17, further comprising at least antimicrobial agent pump configured to be fluidly connected to a source of the antimicrobial agent, wherein the controller is communicatively coupled to the at least one antimicrobial agent pump and configured to control the at least one antimicrobial agent pump based on the concentration of the antimicrobial agent determined.
